(19) United States
(12) Patent Application Publication
(10) Pub. No.: US 2024/0358701 A1
(43) Pub. Date: Oct. 31, 2024

(54) PHARMACEUTICAL COMPOSITIONS AND COMBINATIONS COMPRISING INHIBITORS OF THE ANDROGEN RECEPTOR AND USES THEREOF

(71) Applicant: ESSA Pharma Inc., Vancouver (CA)

(72) Inventor: Peter VIRSIK, Portola Valley, CA (US)

(21) Appl. No.: 18/688,075

(22) PCT Filed: Sep. 1, 2022
(86) PCT No.: PCT/US2022/075838

§ 371 Date: Feb. 29, 2024

Related U.S. Application Data
(60) Provisional application No. 63/355,620, filed on Jun. 26, 2022.
(60) Provisional application No. 63/239,718, filed on Sep. 1, 2021.

(51) Int. Cl. A61K 31/505; A61K 31/4166

(52) U.S. Cl. CPC A61K 31/505; A61K 31/4166

(57) ABSTRACT

The present disclosure generally relates to pharmaceutical compositions and combinations comprising N-(4-((4-(2-(3-chloro-4-(2-chloroethoxy)-5-cyanophenyl)propan-2-yl)phenoxy) methyl)pyrimidin-2-yl)methanesulfonamide or a pharmaceutically acceptable salt, or solvate thereof, and a second therapeutically active agent, such as enzalutamide. In particular, the present disclosure relates to pharmaceutical compositions and combinations useful for treatment of various cancers, for example breast cancer and prostate cancer.

### CROSS-REFERENCE TO RELATED APPLICATIONS

This application claims the benefit of and priority to U.S. Provisional Application No. 63/239,718 filed Sep. 1, 2021 and U.S. Provisional Application No. 63/355,620 filed Jun. 26, 2022, each disclosure is hereby incorporated by reference in its entirety.

### FIELD OF THE INVENTION

The present disclosure generally relates to pharmaceutical compositions and combinations comprising an androgen receptor (AR) N-terminal domain inhibitor (NTD) Compound A and an additional therapeutic agent. In particular, the present disclosure relates to pharmaceutical compositions and combinations useful for treatment of various cancers, such as prostate cancer.

### BACKGROUND OF THE INVENTION

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration also known as androgen ablation therapy (ABT) or androgen deprivation therapy (ADT).

The only effective treatment available for advanced prostate cancer is the withdrawal of androgens which are essential for the survival of prostate luminal cells. Androgen ablation therapy causes a temporary reduction in tumor burden concomitant with a decrease in serum prostate-specific antigen (PSA). Unfortunately, prostate cancer can eventually grow again in the absence of testicular androgens (castration-resistant disease) (Huber et al 1987 Scand J. Urol Nephrol. 104, 33-39). Castration-resistant prostate cancer that is still driven by AR is biochemically characterized before the onset of symptoms by a rising titre of serum PSA (Miller et al 1992 J. Urol. 147, 956-961). Once the disease becomes castration-resistant most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains two transcriptional activation units (tau1 and tau5) within activation function-1 (AF-1). Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et al 1994 Cancer Res. 54, 5474-5478; Nazareth et al 1996 J. Biol. Chem. 271, 19900-19907; Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et al 2002 A J. Biol. Chem. 277, 7076-7085; and Ueda et al2002 B J. Biol. Chem. 277, 38087-38094). The AR can be activated in the absence of testicular androgens by alternative signal transduction pathways in castration-resistant disease, which is consistent with the finding that nuclear AR protein is present in secondary prostate cancer tumors (Kim et al 2002 Am. J. Pathol. 160, 219-226; and van der Kwast et al 1991 Inter. J. Cancer 48, 189-193).

Clinically available inhibitors of the AR include nonsteroidal antiandrogens such as bicalutamide (Casodex™), nilutamide (Anandron®, Nilandron®), flutamide (Eulexin®), enzalutamide (Xtandi®), apalutamide (Erleada®), and darolutamide (Nubega®). There is also a class of steroidal antiandrogens, such as cyproterone acetate and spironolactone. Both steroidal and non-steroidal antiandrogens target the LBD of the AR and predominantly fail presumably due to poor affinity and mutations that lead to activation of the AR by these same antiandrogens (Taplin, M. E., Bubley, G. J., Kom Y. J., Small E. J., Uptonm M., Rajeshkumarm B., Balkm S. P., Cancer Res., 59, 2511-2515 (1999)), and constitutively active AR splice variants. Antiandrogens have no effect on the constitutively active AR splice variants that lack the ligand-binding domain (LBD) and are associated with castration-recurrent prostate cancer (Dehm S M, Schmidt L J, Heemers H V, Vessella R L, Tindall D J., Cancer Res 68, 5469-77, 2008; Guo Z, Yang X, Sun F, Jiang R, Linn D E, Chen H, Chen H, Kong X, Melamed J, Tepper C G, Kung H J, Brodie A M, Edwards J, Qiu Y., Cancer Res. 69, 2305-13, 2009; Hu et al 2009 Cancer Res. 69, 16-22; Sun et al 2010 J Clin Invest. 2010 120, 2715-30) and resistant to abiraterone and enzalutamide (Antonarakis et al., N Engl J Med. 2014, 371, 1028-38; Scher et al JAMA Oncol. 2016 doi: 10.1001). Conventional therapy has concentrated on androgen-dependent activation of the AR through its C-terminal domain.

The AR-NTD is also a target for drug development (e.g. WO 2000/001813; Myung et al. J Clin. Invest 2013, 123, 2948), since the NTD contains Activation-Function-1 (AF-1) which is the essential region required for AR transcriptional activity (Jenster et al 1991. Mol Endocrinol. 5, 1396-404). The AR-NTD importantly plays a role in activation of the AR in the absence of androgens (Sadar, M. D. 1999 J. Biol. Chem. 274, 7777-7783; Sadar M D et al 1999 Endocr Relat Cancer. 6, 487-502; Ueda et al 2002 J. Biol. Chem. 277, 7076-7085; Ueda 2002 J. Biol. Chem. 277, 38087-38094; Blaszczyk et al 2004 Clin Cancer Res. 10, 1860-9; Dehm et al 2006 J Biol Chem. 28, 27882-93; Gregory et al 2004 J Biol Chem. 279, 7119-30). The AR-NTD is important in hormonal progression of prostate cancer as shown by application of decoy molecules (Quayle et al 2007, Proc Natl Acad Sci USA. 104, 1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches. Transcriptionally active androgen receptor plays a major role in CRPC in spite of reduced blood levels of androgen (Karantanos, T. et al Oncogene 2013, 32, 5501-5511; Harris, W. P. et al Nature Clinical Practice Urology, 2009, 6, 76-85). AR mechanisms of resistance to ADT include: overexpression of AR (Visakorpi, T. et al Nature Genetics 1995, 9, 401-406; Koivisto, P. et al Scandinavian Journal of Clinical and Laboratory Investigation Supplementum 1996, 226, 57-63); gain-of-function mutations in the AR LBD (Culig Z. et al Molecular Endocrinology 1993, 7, 1541-1550); intratumoral androgen synthesis (Cai, C. et al Cancer Research 2011, 71, 6503-6513); altered expression and function of AR coactivators (Ueda, T. et al The Journal of Biological Chemistry 2002, 277, 38087-38094; Xu J. et al Nature Reviews Cancer 2009, 9, 615-630); aberrant post-translational modifications of AR (Gioeli D. et al Molecular and Cellular Endocrinology 2012, 352, 70-78; van der Steen T. et al International Journal of Molecular Sciences 2013, 14, 14833-14859); and expression of AR splice variants (AR-Vs) which lack the ligand-binding domain (LBD) (Karantanos, T. et al Oncogene 2013, 32, 5501-5511; Andersen R. J. et al Cancer Cell 2010, 17, 535-546; Myung J. K. et al The Journal of Clinical Investigation 2013, 123, 2948-2960; Sun S. et al The Journal of Clinical Investigation 2010, 120, 2715-2730). Anti-androgens such as bicalutamide and enzalutamide target AR LBD, but have no effect on truncated constitutively active AR-Vs such as AR-V7 (Li Y. et al Cancer Research 2013, 73, 483-489). Expression of AR-V7 is associated with resistance to current hormone therapies (Li Y. et al Cancer Research 2013, 73, 483-489; Antonarakis E. S. et al The New England Journal of Medicine 2014, 371, 1028-1038).

While significant advances have been made in this field, there remains a need for improved treatment for AR-mediated disorders including prostate cancer.

### SUMMARY OF THE INVENTION

The present disclosure relates to pharmaceutical compositions and combinations comprising Compound A and enzalutamide.

The present disclosure relates to a pharmaceutical combination comprising a therapeutically effective amount of a first therapeutically active agent Compound A or a pharmaceutically acceptable salt, or solvate thereof, and a second therapeutically active agent, enzalutamide, in at least one pharmaceutical composition.

In one embodiment of the pharmaceutical combination of the present disclosure, the combination of Compound A and enzalutamide is in a single dosage form. In one embodiment, the combination of Compound A and enzalutamide is in at least two dosage forms.

In one embodiment, the at least two dosage forms are co-packaged together into a single kit.

In another embodiment of the pharmaceutical combinations, the daily dosage amount of Compound A is between about 50 mg and about 1500 mg, or between about 100 mg and about 1000 mg, or between about 200 mg and about 800 mg, or between about 300 mg and about 600 mg. In a specific embodiment, Compound A is included in at least one tablet and the amount of Compound A per tablet is between about 5 mg and about 1000 mg, or between about 10 mg and about 500 mg, or between about 20 mg and about 400 mg, or between about 75 mg and about 300 mg, or between about 150 mg and about 250 mg. In another embodiment, Compound A is included in at least one tablet and amount of Compound A per tablet is about 200 mg.

In another embodiment, the daily dosage amount, or amount per dosage of enzalutamide is about 20 mg to about 500 mg, or about 40 mg to about 250 mg, or about 75 mg to about 300 mg, or about 100 mg to about 200 mg, or about 110 mg to about 170 mg. In a specific embodiment, the daily dosage amount or amount per dosage of enzalutamide is about 120 mg or about 160 mg. As used herein, “per dosage” means at a given time of administration which can be accomplished by one dosage form or multiple dosage forms. For example, in a 160 mg three times a day dosing schedule, 160 mg is administered “per dosage” and it can be with four 40 mg dosage forms.

In another embodiment, a pharmaceutical composition of enzalutamide comprises about 5 mg to about 200 mg enzalutamide, or about 20 mg to about 100 mg enzalutamide, or about 30 mg to about 80 mg enzalutamide, or about 40 mg to about 60 mg enzalutamide. In a specific embodiment, the pharmaceutical composition comprises about 40 mg of enzalutamide. In another embodiment, the composition comprising enzalutamide is an oral capsule.

In a specific embodiment of the pharmaceutical combinations described herein, the pharmaceutical composition may be a tablet or an oral capsule. In a specific embodiment, Compound A and enzalutamide and are in the same composition. In another embodiment, Compound A and enzalutamide are in different pharmaceutical compositions as a kit. In another embodiment, the kit comprises 1 to 6 compositions for each therapeutically active agent to be administered per day.

In another embodiment the pharmaceutical combinations, when administered to a subject, do not produce any serious adverse event to the subject. In a specific embodiment, the serious adverse event is as determined by the U.S Food & Drug Administration. In a specific embodiment, the serious adverse event is determined as provided https://www.fda.gov/safety/reporting-serious-problems-fda/what-serious-adverse-event. In a specific embodiment, administration of said combinations does not produce a treatment-emergent adverse event (TEAE) of Grade 3 or less or a treatment-emergent serious adverse event (TESAE) of Grade 3 or less.

The present disclosure also relates to a method for modulating androgen receptor activity, comprising administering any one of the pharmaceutical combinations and/or compositions as disclosed herein, to a subject in need thereof. In one embodiment, the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

The present disclosure also relates to a method for treating cancer, comprising administering any one of the pharmaceutical combinations and/or compositions as disclosed herein, to a subject in need thereof. In one embodiment, cancer is selected from prostate cancer, breast cancer, ovarian cancer, bladder cancer, pancreatic cancer, hepatocellular cancer, endometrial cancer, or salivary gland carcinoma.

In one embodiment of the methods of the present disclosure, the method is for treating prostate cancer. In one embodiment, prostate cancer is primary or localized prostate cancer, locally advanced prostate cancer, recurrent prostate cancer, advanced prostate cancer, metastatic prostate cancer, metastatic castration-resistant prostate cancer, non-metastatic castration-resistant prostate cancer, and hormone-sensitive prostate cancer. In one embodiment, the prostate cancer is metastatic castration-resistant prostate cancer. In one embodiment, the prostate cancer expresses full-length androgen receptor or truncated androgen receptor splice variant.

### BRIEF DESCRIPTION OF THE DRAWINGS

FIG. 1A shows longitudinal prostate-specific antigen (PSA) changes by patient no. 1 of Cohort 1 (600 mg QD Compound A and 120 mg enzalutamide) of Example 3.

FIG. 1B shows longitudinal prostate-specific antigen (PSA) changes by patient no. 2 of Cohort 1 (600 mg QD Compound A and 120 mg enzalutamide) of Example 3.

FIG. 1C shows longitudinal prostate-specific antigen (PSA) changes by patient no. 3 of Cohort 1 (600 mg QD Compound A and 120 mg enzalutamide) of Example 3.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “an androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

A “prodrug” refers to a derivative of a compound of the present disclosure that will be converted to the compound in vivo. In one embodiment of the present disclosure, a prodrug includes a compound of for example abiraterone having a free hydroxyl group (—OH) that is acetylated (—OCOMe) or acylated at one or more positions.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The term “combination therapy” refers to a first therapy that includes Compound A in conjunction with enzalutamide useful for treating, stabilizing, preventing, and/or delaying the disease or condition.

Administration in “conjunction with” another therapeutically active agent includes administration in the same or different composition(s) and/or combinations, either sequentially, simultaneously, or continuously, through the same or different routes. In some embodiments, the combination therapy optionally includes one or more pharmaceutically acceptable carriers or excipients, non-pharmaceutically active compounds, and/or inert substances.

The terms “pharmaceutical combination,” “therapeutic combination” or “combination” as used herein, refers to a single dosage form comprising at least two therapeutically active agents, or separate dosage forms comprising at least two therapeutically active agents together or separately for use in a combination therapy. For example, one therapeutically active agent may be formulated into one dosage form and the other therapeutically active agent may be formulated into a single or different dosage forms. In a specific example, one therapeutically active agent may be formulated into a solid oral dosage form whereas the second therapeutically active agent may be formulated into a solution dosage form for parenteral administration, including as a kit, or from two kits.

A “co-packaged form” as used herein means that the therapeutically active agents are taken together, more than one dosage forms wherein the therapeutically active agents are taken together, or more than one dosage forms wherein the therapeutically active agents are taken separately in two or more pharmaceutical compositions, i.e., such as two or more separate tablets, capsules, gel capsules, pellets, etc, but typically the separate compositions are as a single kit.

As used herein, the term “pharmaceutical composition” refers to a formulation comprising at least one therapeutically active agent and a pharmaceutically acceptable excipient or carrier. A non-limiting example of pharmaceutical compositions includes tablets, capsules, gel capsules, syrup, liquid, gel, suspension, solid dispersion, or combinations thereof.

As used herein, the term “dosage form” refers to one or more pharmaceutical compositions which provides a specific amount of a therapeutically active agent, such as a unit dose. In one embodiment, a dosage form can be provided in one or more pharmaceutical compositions. For example, if a subject is to be administered with 200 mg of a therapeutically active agent at a time (unit dose), a dosage form can comprise two tablets each containing 100 mg of the therapeutically active agent, wherein the two tablets are the same pharmaceutical composition.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof

The following description includes information that may be useful in understanding the present invention. It is not an admission that any of the information provided herein is prior art or relevant to the presently claimed inventions, or that any publication specifically or implicitly referenced is prior art.

### Pharmaceutical Combination of the Disclosure

The present disclosure relates to pharmaceutical combinations and/or compositions comprising Compound A and enzalutamide. In one embodiment, the pharmaceutical combinations and/or compositions of the present disclosure is useful for treating various diseases and conditions including, but not limited to, cancer. In one embodiment, the pharmaceutical combinations and/or compositions of the present disclosure is useful for treating prostate cancer.

### Compound A

The present disclosure relates to pharmaceutical compositions or combinations comprising N-(4-((4-(2-(3-chloro-4-(2-chloroethoxy)-5-cyanophenyl)propan-2-yl)phenoxy) methyl)pyrimidin-2-yl)methanesulfonamide (Compound A), or a pharmaceutically acceptable salt, or solvate thereof. Compound A has the following structure:

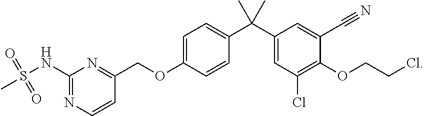 [SMILES: CC(C)(c1ccc(OCc2ccnc(NS(C)(=O)=O)n2)cc1)c1cc(Cl)c(OCCCl)c(C#N)c1]

Compound A is an androgen receptor modulator. Compound A binds to androgen receptor. Specifically, Compound A is an androgen receptor N-terminal domain inhibitor.

### Androgen Receptor Ligand-Binding Domain Inhibitors

In one embodiment, the additional therapeutically active agent is an androgen receptor ligand-binding domain inhibitor.

In one embodiment, the androgen receptor ligand-binding domain inhibitor is enzalutamide.

### Representative Combination

The present disclosure relates to pharmaceutical compositions and combinations comprising Compound A or a pharmaceutically acceptable salt or a solvate and enzalutamide. In one embodiment, the composition and/or combination comprises a third therapeutically active agent. The present disclosure relates to a pharmaceutical combination comprising a therapeutically effective amount of a first therapeutically active agent Compound A or a pharmaceutically acceptable salt or solvate thereof, and enzalutamide in at least one pharmaceutical composition.

In one embodiment, the pharmaceutical combination relates to Compound A or a pharmaceutically acceptable salt, or solvate thereof, and enzalutamide.

### Therapeutic Use

The pharmaceutical compositions and combinations of the present disclosure find use in any number of methods. For example, in some embodiments the compounds are useful in methods for modulating androgen receptor (AR). In some embodiments, modulating androgen receptor (AR) activity is in a mammalian cell. In some embodiments, modulating androgen receptor (AR) can be in a subject in need thereof (e.g., a mammalian subject) and for treatment of any of the described conditions or diseases.

In one embodiment, the modulating AR is binding to AR. In other embodiments, the modulating AR is inhibiting AR.

In one embodiment, the modulating AR is modulating AR N-terminal domain (NTD). In one embodiment, the modulating AR is modulating AR NTD and AR ligand-binding domain (LBD). In one embodiment, the modulating AR is binding to AR NTD. In one embodiment, the modulating AR is binding to AR NTD and AR LBD. In other embodiments, the modulating AR is inhibiting AR NTD. In other embodiments, the modulating AR is inhibiting AR NTD and AR LBD. In some embodiments, modulating the AR is inhibiting transactivation of androgen receptor N-terminal domain (NTD).

In one embodiment of the present disclosure, methods for modulating androgen receptor activity, comprising administering any one of the pharmaceutical combinations and/or compositions as disclosed herein, to a subject in need thereof, are provided. In other embodiments, modulating androgen receptor (AR) activity is for treatment of at least one indication selected from the group consisting of: prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof. For example, in some embodiments, the indication is prostate cancer. In other embodiments, the prostate cancer is primary/localized prostate cancer, locally advanced prostate cancer, recurrent prostate cancer, metastatic prostate cancer, advanced prostate cancer, or metastatic castration-resistant prostate cancer (mCRPC), or hormone-sensitive prostate cancer. In other embodiments, the prostate cancer is non-metastatic castration-resistant prostate cancer (nmCRPC). While in other embodiments, the prostate cancer is androgen dependent prostate cancer. In other embodiments, the spinal and bulbar muscular atrophy is Kennedy's disease.

In one embodiment of the present disclosure, a method of treating a condition associated with cell proliferation in a patient in need thereof is provided. In one embodiment, the present invention provides a method of treating cancer or tumors, comprising administering any one of the pharmaceutical combinations and/or compositions as disclosed herein, to a subject in need thereof. In one embodiment, cancer is selected from prostate cancer, breast cancer, ovarian cancer, bladder cancer, pancreatic cancer, hepatocellular cancer, endometrial cancer, or salivary gland carcinoma.

In one embodiment of the methods of the present disclosure, the method is for treating prostate cancer. In one embodiment, prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer. In one embodiment, prostate cancer is primary or localized prostate cancer, locally advanced prostate cancer, recurrent prostate cancer, advanced prostate cancer, metastatic prostate cancer, metastatic castration-resistant prostate cancer, non-metastatic castration-resistant prostate cancer, and hormone-sensitive prostate cancer. In one embodiment, the prostate cancer is metastatic castration-resistant prostate cancer. In one embodiment, the prostate cancer is non-metastatic castration-resistant prostate cancer. In one embodiment, the prostate cancer expresses full-length androgen receptor or truncated androgen receptor splice variant. In one embodiment, the prostate cancer is resistant to enzalutamide monotherapy.

In one embodiment of the present disclosure, a method of reducing, inhibiting, or ameliorating cell proliferation in a patient in need thereof is provided. In one embodiment, the reducing, inhibiting, or ameliorating in the method disclosed herein, is in vivo. In another embodiment, the reducing, inhibiting, or ameliorating is in vitro.

In one embodiment, the cells in the method disclosed herein, are cancer cells. In one embodiment, the cancer cells are prostate cancer cells. In one embodiment, the prostate cancer cells are cells of primary/localized prostate cancer (newly diagnosed or early stage), locally advanced prostate cancer, recurrent prostate cancer (e.g., prostate cancer which was not cured with primary therapy), metastatic prostate cancer, advanced prostate cancer (e.g., after castration for recurrent prostate cancer), metastatic castration-resistant prostate cancer (mCRPC), or hormone-sensitive prostate cancer. In one embodiment, the prostate cancer cells are cells of primary/localized prostate cancer, locally advanced prostate cancer, recurrent prostate cancer, metastatic prostate cancer, advanced prostate cancer, metastatic castration-resistant prostate cancer (mCRPC), non-metastatic castration-resistant prostate cancer (nmCRPC), or hormone-sensitive prostate cancer. In one embodiment, the prostate cancer cells are cells of non-metastatic castration-resistant prostate cancer (nmCRPC). In another embodiment, the prostate cancer cells are cells of a metastatic castration-resistant prostate cancer. In other embodiments, the prostate cancer cells are androgen-dependent prostate cancer cells or androgen-independent prostate cancer cells.

In one embodiment, the condition or disease associated with cell proliferation is cancer. In one embodiment of any one of the methods disclosed herein, the cancer is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In one embodiment, the condition or disease is prostate cancer. In one embodiment, prostate cancer is selected from primary/localized prostate cancer, locally advanced prostate cancer, recurrent prostate cancer, metastatic prostate cancer, advanced prostate cancer, metastatic castration-resistant prostate cancer (mCRPC), or hormone-sensitive prostate cancer. In one embodiment, prostate cancer is selected from primary/localized prostate cancer, locally advanced prostate cancer, recurrent prostate cancer, metastatic prostate cancer, advanced prostate cancer, metastatic castration-resistant prostate cancer (mCRPC), non-metastatic castration-resistant prostate cancer (nmCRPC), or hormone-sensitive prostate cancer. In another embodiment, the prostate cancer is a metastatic castration-resistant prostate cancer. In another embodiment, the prostate cancer is a non-metastatic castration-resistant prostate cancer. In some embodiments, the prostate cancer is an androgen-dependent prostate cancer cells or an androgen-independent prostate cancer. In one embodiment, the condition or disease is breast cancer. In one embodiment, the breast cancer is AR-positive triple negative breast cancer.

In some embodiments, the subject has mCRPC and the mCRPC subject is naïve to one or more second generation anti-androgens. In some embodiments, the second generation anti-androgen is abiraterone acetate, enzalutamide, apalutamide and/or darolutamide. In some embodiments, the subject has received no prior chemotherapy before administration. In some embodiments, the subject has received prior chemotherapy before administration.

In another embodiment of the present disclosure, a method for reducing or preventing tumor growth, comprising contacting tumor cells with a pharmaceutical composition or a combination as disclosed herein.

In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth. In one embodiment, reducing or preventing tumor growth includes reduction in the rate of tumor growth such that the rate of tumor growth before treating a patient with the methods disclosed herein (r1) is faster than the rate of tumor growth after said treatment (r2) such that r1>r2.

In one embodiment, the reducing or preventing in the methods disclosed herein is in vivo. In another embodiment, the treating is in vitro.

In one embodiment, the tumor cell in the method disclosed herein is selected from prostate cancer, breast cancer, ovarian cancer, endometrial cancer, or salivary gland carcinoma. In one embodiment, the tumor cells are prostate cancer tumor cells. In one embodiment, the prostate cancer tumor cells are tumor cells of primary/localized prostate cancer, locally advanced prostate cancer, recurrent prostate cancer, metastatic prostate cancer, advanced prostate cancer, metastatic castration-resistant prostate cancer (mCRPC), or hormone-sensitive prostate cancer. In one embodiment, the prostate cancer tumor cells are tumor cells of primary/localized prostate cancer, locally advanced prostate cancer, recurrent prostate cancer, metastatic prostate cancer, advanced prostate cancer, metastatic castration-resistant prostate cancer (mCRPC), non-metastatic castration-resistant prostate cancer (nmCRPC), or hormone-sensitive prostate cancer. In other embodiments, the prostate cancer is a metastatic castration-resistant prostate cancer. In other embodiments, the prostate cancer is a non-metastatic castration-resistant prostate cancer. In some embodiments, the prostate cancer is androgen-dependent prostate cancer or androgen-independent prostate cancer. In another embodiment, the tumor cells are is breast cancer tumor cells.

In one embodiment of the methods of the present disclosure, Compound A is administered at a daily dosage amount between about 50 mg and about 1500 mg, or between about 100 mg and about 1000 mg, or between about 200 mg and about 800 mg, or between about 300 mg and about 600 mg, or any values or subranges therebetween. In one embodiment, the daily dosage amount of Compound A is about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, or about 1500 mg, or any values therebetween. In one embodiment, the daily dose of Compound A is administered once a day or divided into twice-a-day or three times a day doses. In one embodiment, the daily dose of Compound A is provided in one tablet or one capsule, or the daily dose is divided into two, three, four, five, or six tablets or capsules.

In one embodiment of the methods of the present disclosure, Compound A is administered at a daily dosage amount of about 100 mg QD (once a day), about 150 mg QD, about 200 mg QD, about 250 mg QD, about 300 mg QD, about 350 mg QD, about 400 mg QD, about 450 mg QD, about 500 mg QD, about 550 mg QD, about 600 mg QD, about 650 mg QD, about 700 mg QD, about 750 mg QD, about 800 mg QD, about 850 mg QD, about 900 mg QD, about 950 mg QD, about 1,000 mg QD, about 1,050 mg QD, about 1,100 mg QD, about 1,150 mg QD, or about 1,200 mg QD, or any values therebetween. In one embodiment of the methods of the present disclosure, Compound A is administered at a daily dose of about 200 mg QD, about 400 mg QD, about 600 mg QD, about 800 mg QD, or about 1,000 mg QD, or any values therebetween.

In one embodiment of the methods of the present disclosure, Compound A is administered at a daily dose of about 100 mg BID (twice a day), about 150 mg BID, about 200 mg BID, about 250 mg BID, about 300 mg BID, about 350 mg BID, about 400 mg BID, about 450 mg BID, about 500 mg BID, about 550 mg BID, about 600 mg BID, about 650 mg BID, about 700 mg BID, about 750 mg BID, about 800 mg BID, about 850 mg BID, about 900 mg BID, about 950 mg BID, about 1,000 mg BID, about 1,050 mg BID, about 1,100 mg BID, about 1,150 mg BID, or about 1,200 mg BID, or any values therebetween. In one embodiment of the methods of the present disclosure, Compound A is administered at a daily dose of about 400 mg BID, about 600 mg BID, or about 800 mg BID, or any values therebetween.

In some embodiments of the methods of the present disclosure, the pharmaceutical combination is administered to a subject for at least one week, two weeks, three weeks, four weeks, five weeks, six weeks, seven weeks, eight weeks, nine weeks, or ten weeks.

In some embodiments of the methods of the present disclosure, the pharmaceutical combination is administered to a subject for at least one cycle, two cycles, three cycles, four cycles, five cycles, six cycles, seven cycles, eight cycles, nine cycles, or ten cycles. In some embodiments, one cycle is about 7 days, about 14 days, about 21 days, about 28 days, or about 35 days. In some embodiments, one cycle is about 28 days.

In some embodiments of the methods of the present disclosure, the pharmaceutical combination of the present disclosure provides PSA90 (prostate-specific antigen decline of >90%) response rate of about 30% or greater, about 40% or greater, about 50% or greater, about 60% or greater, about 65% or greater, about 70% or greater, or about 75% or greater, including all values therebetween. In some embodiments of the methods of the present disclosure, the pharmaceutical combination of the present disclosure provides PSA90 response rate of about 60% or greater or about 65% or greater.

In some embodiments of the methods of the present disclosure, the pharmaceutical combination of the present disclosure provides PSA50 (PSA decline of >50%) response rate of about 30% or greater, about 40% or greater, about 50% or greater, about 60% or greater, about 65% or greater, about 70% or greater, about 75% or greater, about 80% or greater, about 85% or greater, or about 90% or greater, including all values therebetween. In some embodiments of the methods of the present disclosure, the pharmaceutical combination of the present disclosure provides PSA50 response rate of about 60% or greater, about 70% or greater, or about 80% or greater.

In some embodiments of the methods of the present disclosure, the pharmaceutical combination of the present disclosure after administration to the subject provides a PSA90 (prostate-specific antigen decline of >90%) within about 8 weeks, within about 7 weeks, within about 6 weeks, within about 5 weeks, within about 4 weeks, within about 3 weeks, within about 2 weeks or within about 1 week.

In some embodiments of the methods of the present disclosure, the pharmaceutical combination of the present disclosure after administration to the subject provides a PSA90 (prostate-specific antigen decline of >50%) within about 8 weeks, within about 7 weeks, within about 6 weeks, within about 5 weeks, within about 4 weeks, within about 3 weeks, within about 2 weeks, within about 13 days, within about 12 days, within about 11 days, within about 10 days, within about 9 days within about 8 days, within about 7 days, within about 6 days, or within about 5 days.

In some embodiments of the methods of the present disclosure, the pharmaceutical combination of the present disclosure provides PSA<0.2 ng/mL rate of about 10% or greater, about 15% or greater, about 20% or greater, about 25% or greater, about 30% or greater, about 35% or greater, or about 40% or greater, including all values therebetween. In some embodiments of the methods of the present disclosure, the pharmaceutical combination of the present disclosure provides PSA<0.2 ng/mL rate of about 25% or greater or about 30% or greater.

In some embodiments of the methods of the present disclosure, the pharmaceutical combination of the present disclosure provides AUCss (area under the concentration-time curve at steady state) for Compound A in the range of about 20,000 ng·hr/mL to about 500,000 ng·hr/mL, including all subranges and values therebetween. In some embodiments of the methods of the present disclosure, the pharmaceutical combination of the present disclosure provides AUCss for Compound A in the range of about 30,000 ng·hr/mL to about 450,000 ng·hr/mL, including all subranges and values therebetween. In some embodiments of the methods of the present disclosure, the pharmaceutical combination of the present disclosure provides AUCss for Compound A in the range of about 50,000 ng·hr/mL to about 400,000 ng·hr/mL, including all subranges and values therebetween. In some embodiments of the methods of the present disclosure, the pharmaceutical combination of the present disclosure provides AUCss for Compound A in the range of about 60,000 ng·hr/mL to about 350,000 ng·hr/mL, including all subranges and values therebetween. In some embodiments of the methods of the present disclosure, the pharmaceutical combination of the present disclosure provides AUCss for Compound A in the range of about 75,000 ng·hr/mL to about 300,000 ng·hr/mL, including all subranges and values therebetween. In some embodiments of the methods of the present disclosure, the pharmaceutical combination of the present disclosure provides AUCss for Compound A in the range of about 100,000 ng·hr/mL to about 300,000 ng·hr/mL, including all subranges and values therebetween. In some embodiments of the methods of the present disclosure, the pharmaceutical combination of the present disclosure provides AUCss for Compound A in the range of about 150,000 ng·hr/mL to about 300,000 ng·hr/mL, including all subranges and values therebetween. In some embodiments of the methods of the present disclosure, the pharmaceutical combination of the present disclosure provides AUCss for Compound A in the range of about 175,000 ng·hr/mL to about 275,000 ng·hr/mL, including all subranges and values therebetween. In some embodiments of the methods of the present disclosure, the pharmaceutical combination of the present disclosure provides AUCss for Compound A in the range of about 200,000 ng·hr/mL to about 275,000 ng·hr/mL, including all subranges and values therebetween. In some embodiments, the AUCss for Compound A as described herein is for a subject who had no prior chemotherapy treatment before receiving the combination treatment of the present disclosure. In some embodiments, AUCss for Compound A is measured in the subject's blood or plasma.

In some embodiments of the methods of the present disclosure, the pharmaceutical combination of the present disclosure provides AUCss (area under the concentration-time curve at steady state) for Compound A in the range of about 20,000 ng·hr/mL to about 300,000 ng·hr/mL, including all subranges and values therebetween. In some embodiments of the methods of the present disclosure, the pharmaceutical combination of the present disclosure provides AUCss for Compound A in the range of about 30,000 ng·hr/mL to about 250,000 ng·hr/mL, including all subranges and values therebetween. In some embodiments of the methods of the present disclosure, the pharmaceutical combination of the present disclosure provides AUCss for Compound A in the range of about 40,000 ng·hr/mL to about 200,000 ng·hr/mL, including all subranges and values therebetween. In some embodiments of the methods of the present disclosure, the pharmaceutical combination of the present disclosure provides AUCss for Compound A in the range of about 40,000 ng·hr/mL to about 150,000 ng·hr/mL, including all subranges and values therebetween. In some embodiments of the methods of the present disclosure, the pharmaceutical combination of the present disclosure provides AUCss for Compound A in the range of about 40,000 ng·hr/mL to about 100,000 ng·hr/mL, including all subranges and values therebetween. In some embodiments of the methods of the present disclosure, the pharmaceutical combination of the present disclosure provides AUCss for Compound A in the range of about 40,000 ng·hr/mL to about 80,000 ng·hr/mL, including all subranges and values therebetween. In some embodiments, the AUCss for Compound A as described herein is for a subject who had received prior chemotherapy treatment before receiving the combination treatment of the present disclosure. In some embodiments, AUCss for Compound A is measured in the subject's blood or plasma.

In some embodiments of the methods of the present disclosure, the pharmaceutical combination of the present disclosure provides AUCss for enzalutamide and its active metabolite N-desmethyl enzalutamide (M2 metabolite) in the range of about 100,000 ng·hr/mL to about 1,000,000 ng·hr/mL, including all subranges and values therebetween. In some embodiments of the methods of the present disclosure, the pharmaceutical combination of the present disclosure provides AUCss for enzalutamide+M2 metabolite in the range of about 150,000 ng·hr/mL to about 950,000 ng·hr/mL, including all subranges and values therebetween. In some embodiments of the methods of the present disclosure, the pharmaceutical combination of the present disclosure provides AUCss for enzalutamide+M2 metabolite in the range of about 200,000 ng·hr/mL to about 900,000 ng·hr/mL, including all subranges and values therebetween. In some embodiments of the methods of the present disclosure, the pharmaceutical combination of the present disclosure provides AUCss for enzalutamide+M2 metabolite in the range of about 250,000 ng·hr/mL to about 850,000 ng·hr/mL, including all subranges and values therebetween. In some embodiments of the methods of the present disclosure, the pharmaceutical combination of the present disclosure provides AUCss for enzalutamide+M2 metabolite in the range of about 300,000 ng·hr/mL to about 800,000 ng·hr/mL, including all subranges and values therebetween. In some embodiments of the methods of the present disclosure, the pharmaceutical combination of the present disclosure provides AUCss for enzalutamide+M2 metabolite in the range of about 350,000 ng·hr/mL to about 750,000 ng·hr/mL, including all subranges and values therebetween. In some embodiments of the methods of the present disclosure, the pharmaceutical combination of the present disclosure provides AUCss for enzalutamide+M2 metabolite in the range of about 400,000 ng·hr/mL to about 700,000 ng·hr/mL, including all subranges and values therebetween. In some embodiments of the methods of the present disclosure, the pharmaceutical combination of the present disclosure provides AUCss for enzalutamide+M2 metabolite in the range of about 450,000 ng·hr/mL to about 650,000 ng·hr/mL, including all subranges and values therebetween. In some embodiments of the methods of the present disclosure, the pharmaceutical combination of the present disclosure provides AUCss for enzalutamide+M2 metabolite in the range of about 500,000 ng·hr/mL to about 600,000 ng·hr/mL, including all subranges and values therebetween. In some embodiments, the AUCss for enzalutamide+M2 metabolite as described herein is for a subject who had no prior chemotherapy treatment before receiving the combination treatment of the present disclosure. In some embodiments, AUCss for enzalutamide+M2 metabolite is measured in the subject's blood or plasma.

In some embodiments of the methods of the present disclosure, the pharmaceutical combination of the present disclosure provides AUCss for enzalutamide and its active metabolite N-desmethyl enzalutamide (M2 metabolite) in the range of about 100,000 ng·hr/mL to about 800,000 ng·hr/mL, including all subranges and values therebetween. In some embodiments of the methods of the present disclosure, the pharmaceutical combination of the present disclosure provides AUCss for enzalutamide+M2 metabolite in the range of about 150,000 ng·hr/mL to about 750,000 ng·hr/mL, including all subranges and values therebetween. In some embodiments of the methods of the present disclosure, the pharmaceutical combination of the present disclosure provides AUCss for enzalutamide+M2 metabolite in the range of about 200,000 ng·hr/mL to about 700,000 ng·hr/mL, including all subranges and values therebetween. In some embodiments of the methods of the present disclosure, the pharmaceutical combination of the present disclosure provides AUCss for enzalutamide+M2 metabolite in the range of about 250,000 ng·hr/mL to about 650,000 ng·hr/mL, including all subranges and values therebetween. In some embodiments of the methods of the present disclosure, the pharmaceutical combination of the present disclosure provides AUCss for enzalutamide+M2 metabolite in the range of about 300,000 ng·hr/mL to about 600,000 ng·hr/mL, including all subranges and values therebetween. In some embodiments of the methods of the present disclosure, the pharmaceutical combination of the present disclosure provides AUCss for enzalutamide+M2 metabolite in the range of about 350,000 ng·hr/mL to about 550,000 ng·hr/mL, including all subranges and values therebetween. In some embodiments of the methods of the present disclosure, the pharmaceutical combination of the present disclosure provides AUCss for enzalutamide+M2 metabolite in the range of about 350,000 ng·hr/mL to about 500,000 ng·hr/mL, including all subranges and values therebetween. In some embodiments of the methods of the present disclosure, the pharmaceutical combination of the present disclosure provides AUCss for enzalutamide+M2 metabolite in the range of about 400,000 ng·hr/mL to about 500,000 ng·hr/mL, including all subranges and values therebetween. In some embodiments, the AUCss for enzalutamide+M2 metabolite as described herein is for a subject who had received prior chemotherapy treatment before receiving the combination treatment of the present disclosure. In some embodiments, AUCss for enzalutamide+M2 metabolite is measured in the subject's blood or plasma.

In some embodiments of the methods of the present disclosure, the subject received one or more chemotherapy treatment prior to receiving the combination treatment of the present disclosure. In some embodiments of the methods of the present disclosure, the subject received no chemotherapy treatment prior to receiving the combination treatment of the present disclosure.

In some embodiments of the methods of the present disclosure, the subject has histologically, pathologically, or cytologically confirmed prostate adenocarcinoma. In some embodiments of the methods of the present disclosure, the subject has histologically, pathologically, or cytologically confirmed prostate adenocarcinoma without small cell or neuroendocrine features.

In some embodiments of the methods of the present disclosure, the subject has PSA levels≥1 ng/mL while on androgen deprivation therapy (ADT). In some embodiments of the methods of the present disclosure, the subject has PSA levels≥1 ng/mL while on ADT and documentation of three rising PSA levels taken at least 1 week apart during ADT (or surgical castration).

In some embodiments of the methods of the present disclosure, the subject has one or more bone lesions on bone scan or by soft tissue disease observed by CT/MRI.

In some embodiments of the methods of the present disclosure, the subject has nodal or visceral progression.

In some embodiments of the methods of the present disclosure, the subject is naïve to one or more second generation anti-androgens.

In some embodiments of the methods of the present disclosure, the subject is on ADT with luteinizing hormone-releasing hormone (LHRH) agonist/antagonist therapy. In some embodiments, the subject has a history of bilateral orchiectomy with castrate level testosterone.

In some embodiments of the methods of the present disclosure, the subject has serum testosterone≤1.73 nmol/L (50 ng/dL).

### Pharmaceutical Compositions, Formulations, and Dosage Forms

The pharmaceutical combination of the present disclosure can be presented in various forms of pharmaceutical compositions. In one embodiment, the pharmaceutical combination comprising a therapeutically effective amount of a first therapeutically active agent Compound A or a pharmaceutically acceptable salt, or solvate thereof, and enzalutamide is provided in at least one pharmaceutical composition.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. In some embodiments, the compounds are administered orally.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, gel capsules, liquids, gels, syrups, slurries, suspensions and the like, for oral ingestion by a subject.

In one embodiment of the pharmaceutical combination of the present disclosure, the combination of Compound A and enzalutamide is in the same dosage form.

In one embodiment, the combination of Compound A and enzalutamide is provided in at least two dosage forms or at least two pharmaceutical compositions. In one embodiment, the at least two dosage forms or the at least two pharmaceutical compositions are co-packaged together into a single kit. In one embodiment, Compound A is provided in one dosage form or one pharmaceutical composition and enzalutamide is provided in another dosage form or another pharmaceutical composition. In one embodiment, a single kit comprises Compound A in one dosage form or a pharmaceutical composition and enzalutamide in another dosage form or a pharmaceutical composition. In one embodiment, a single kit comprises Compound A formulated into one or more tablets or capsules and enzalutamide in different tablets or capsules. In one embodiment, a single kit comprises Compound A formulated into one or more tablets and enzalutamide in different tablets.

In one embodiment, a single kit comprises a single dose of Compound A and enzalutamide. In one embodiment, a single kit comprises a daily dose of Compound A and enzalutamide. In one embodiment, a daily dose may comprise one or more single doses of Compound A and/or the enzalutamide to be taken at one, two, three, or four different times of the day. In one embodiment, Compound A and enzalutamide has the same dosing frequency (e.g., once a day, twice a day, once a week). In one embodiment, Compound A and enzalutamide has the same dosing frequency but taken at different times of the day. In one embodiment, Compound A and enzalutamide has the same dosing frequency and taken at the same time of the day. In one embodiment, Compound A and enzalutamide has a different dosing frequency (e.g., Compound A is taken once a day and enzalutamide is taken twice a day).

In one embodiment, the combination of Compound A, and enzalutamide is provided in at least two dosage forms or at least two pharmaceutical compositions. In one embodiment, the combination is provided in at least three dosage forms. In one embodiment, the at least two dosage forms or the at least two pharmaceutical compositions are co-packaged together into a single kit. In one embodiment, Compound A is provided in one dosage form or one pharmaceutical composition, and enzalutamide is provided in another dosage form or another pharmaceutical composition. In one embodiment, Compound A and enzalutamide are provided in one dosage form or pharmaceutical composition. In one embodiment, a single kit comprises Compound A in one dosage form or a pharmaceutical composition, and enzalutamide in another dosage form or a pharmaceutical composition. In one embodiment, a single kit comprises Compound A formulated into one or more tablets or capsules, and enzalutamide in different tablets or capsules. In one embodiment, a single kit comprises Compound A formulated into one or more tablets and enzalutamide in different capsules.

In one embodiment of the pharmaceutical combination of the present disclosure, the pharmaceutical combination comprises a kit comprising, one, two or three different dosage forms co-packaged together. Different dosage forms in a single co-package can comprise different therapeutically active agents. In some embodiments, all therapeutically active agents are provided in different dosage forms. In some embodiments, two or more therapeutically active agents are formulated into the same dosage form. In one embodiment, the kit can comprise 1, 2, 3, 4, 5, or 6 pharmaceutical compositions of each dosage form.

In one embodiment of the pharmaceutical combination of the present disclosure, all pharmaceutical compositions are co-packaged for daily administration.

In one embodiment of the pharmaceutical combination of the present disclosure, each pharmaceutical composition of each dosage form is for administration to a subject once every 24 hours, once every 12 hours, once every 8 hours, once every 6 hours, once every 5 hours, or once every 4 hours. In one embodiment, different therapeutically active agents in the combination can have different dosing schedule.

In one embodiment of the pharmaceutical combination of the present disclosure, Compound A and enzalutamide are in different compositions but provided in a single kit. In one embodiment, the kit comprises 1, 2, 3, 4, 5, or 6 compositions for each therapeutically active agent to be administered per day. In one embodiment, the kit comprises 1, 2, 3, 4, 5, or 6 tablets or capsules or mixtures of tablets and capsules for each therapeutically active agent.

In one embodiment of the pharmaceutical combination of the present disclosure, at least one composition is a tablet.

In one embodiment of the pharmaceutical combination of the present disclosure, a daily dosage amount of Compound A is between about 50 mg and about 1500 mg, or between about 100 mg and about 1000 mg, or between about 200 mg and about 800 mg, or between about 300 mg and about 600 mg, or any values or subranges therebetween. In one embodiment, the daily dosage amount of Compound A is about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, or about 1500 mg, or any values therebetween. In one embodiment, the daily dose of Compound A is administered once a day or divided into twice-a-day or three times a day doses. In one embodiment, the daily dose of Compound A is provided in one tablet or one capsule, or the daily dose is divided into two, three, four, five, or six tablets or capsules.

In one embodiment of the pharmaceutical combination of the present disclosure, a daily dosage amount of Compound A is about 100 mg QD (once a day), about 150 mg QD, about 200 mg QD, about 250 mg QD, about 300 mg QD, about 350 mg QD, about 400 mg QD, about 450 mg QD, about 500 mg QD, about 550 mg QD, about 600 mg QD, about 650 mg QD, about 700 mg QD, about 750 mg QD, about 800 mg QD, about 850 mg QD, about 900 mg QD, about 950 mg QD, about 1,000 mg QD, about 1,050 mg QD, about 1,100 mg QD, about 1,150 mg QD, or about 1,200 mg QD, or any values therebetween. In one embodiment of the pharmaceutical combination of the present disclosure, a daily dosage amount of Compound A is about 200 mg QD, about 400 mg QD, about 600 mg QD, about 800 mg QD, or about 1,000 mg QD, or any values therebetween.

In one embodiment of the pharmaceutical combination of the present disclosure, a daily dosage amount of Compound A is about 100 mg BID (twice a day), about 150 mg BID, about 200 mg BID, about 250 mg BID, about 300 mg BID, about 350 mg BID, about 400 mg BID, about 450 mg BID, about 500 mg BID, about 550 mg BID, about 600 mg BID, about 650 mg BID, about 700 mg BID, about 750 mg BID, about 800 mg BID, about 850 mg BID, about 900 mg BID, about 950 mg BID, about 1,000 mg BID, about 1,050 mg BID, about 1,100 mg BID, about 1,150 mg BID, or about 1,200 mg BID, or any values therebetween. In one embodiment of the pharmaceutical combination of the present disclosure, a daily dosage amount of Compound A is about 400 mg BID, about 600 mg BID, or about 800 mg BID, or any values therebetween.

In some embodiments of the pharmaceutical combination of the present disclosure, a daily dosage amount of Compound A is higher than a daily dosage amount of Compound A recommended for monotherapy (Compound A alone). In some embodiments of the pharmaceutical combination of the present disclosure, a daily dosage amount of Compound A is higher than a daily dosage amount of Compound A recommended for monotherapy due to drug-drug interaction between Compound A and enzalutamide. In some embodiments, enzalutamide lowers the exposure of Compound A when administered in a combination. In some embodiments, enzalutamide lowers the exposure of Compound A when administered in a combination because enzalutamide is an induce of CYP3A4.

In one embodiment of the pharmaceutical combination of the present disclosure, an amount of Compound A per a dosage form is between about 5 mg and about 1000 mg, or between about 10 mg and about 500 mg, or between about 20 mg and about 250 mg, or between about 30 mg and about 300 mg, or between about 50 mg and about 200 mg, or any values or subranges therebetween. In one embodiment, an amount of Compound A per a dosage form is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values therebetween.

In one embodiment of the pharmaceutical combination of the present disclosure, an amount of Compound A per one tablet or one capsule is between about 5 mg and about 1,000 mg, or between about 10 mg and about 500 mg, or between about 20 mg and about 250 mg, or between about 30 mg and about 300 mg, or between about 50 mg and about 200 mg, or any values or subranges therebetween. In one embodiment, an amount of Compound A per one tablet or one capsule is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values therebetween.

In one embodiment of the pharmaceutical combination of the present disclosure, the second therapeutically active agent is enzalutamide and a daily dosage amount of enzalutamide is about 25 mg to about 550 mg, or about 50 mg to about 480 mg, or about 100 mg to about 400 mg, or about 120 mg to about 200 mg, or any values or subranges therebetween. In one embodiment, the daily dosage amount of enzalutamide is about 20 mg, about 25 mg, about 30 mg, about 40 mg, about 50 mg, about 60 mg, about 70 mg, about 80 mg, about 90 mg, about 100 mg, about 150 mg, about 160 mg, about 200 mg, about 250 mg, about 300 mg, about 350 mg, about 400 mg, about 450 mg, about 480 mg, about 500 mg, about 550 mg, or about 600 mg, or any values therebetween. In one embodiment, the daily dosage amount of enzalutamide is about 80 mg, about 120 mg, or about 160 mg, or any values therebetween. In one embodiment, the daily dose of enzalutamide is administered once a day or divided into twice-a-day or three times a day. In one embodiment, the daily dose of enzalutamide is provided in one tablet or one capsule, or the daily dose is divided into two, three, four, five, or six tablets or capsules.

In some embodiments of the pharmaceutical combination of the present disclosure, a daily dosage amount of enzalutamide is lower than a daily dosage amount of enzalutamide recommended for monotherapy (enzalutamide alone). In some embodiments of the pharmaceutical combination of the present disclosure, a daily dosage amount of enzalutamide is lower than a daily dosage amount of enzalutamide recommended for monotherapy due to drug-drug interaction between Compound A and enzalutamide. In some embodiments, Compound A increases the exposure of enzalutamide when administered in a combination. In some embodiments, Compound A increases the exposure of enzalutamide when administered in a combination because Compound A is an inhibitor of cytochrome P450 (CYP)2C8.

In one embodiment of the pharmaceutical combination of the present disclosure, wherein enzalutamide per a dosage form is about 5 mg to about 300 mg, or about 10 mg to about 200 mg, or about 30 mg to about 450 mg, or about 120 mg to about 200 mg, or any values or subranges therebetween. In one embodiment, the amount of enzalutamide per a dosage form is about is about 20 mg to about 500 mg, or about 40 mg to about 250 mg, or about 75 mg to about 300 mg, or about 100 mg to about 200 mg, or about 110 mg to about 170 mg, or any values therebetween.

In one embodiment, a pharmaceutical composition of enzalutamide comprises about 5 mg to about 200 mg enzalutamide, or about 20 mg to about 100 mg enzalutamide, or about 30 mg to about 80 mg enzalutamide, or about 40 mg to about 60 mg enzalutamide or any values therebetween.

In one embodiment of the pharmaceutical combination of the present disclosure, an amount of enzalutamide per each tablet or capsule is about 5 mg to about 300 mg, or about 10 mg to about 200 mg, or about 20 mg to about 100 mg, or about 30 mg to about 80 mg, or any values or subranges therebetween. In one embodiment, the amount of enzalutamide per one tablet or one capsule is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg, or any values therebetween. In one embodiment, the amount of enzalutamide per one tablet or one capsule is about 40 mg or about 60 mg.

### Various Embodiments of Pharmaceutical Compositions, Formulations, Dosage Forms

The pharmaceutical composition or a combination as disclosed herein, can further comprise a pharmaceutically acceptable carrier or excipient.

In a further embodiment of the present disclosure, a pharmaceutical composition or combination as disclosed herein comprises a pharmaceutically acceptable carrier, excipient or adjuvant is provided. The pharmaceutically acceptable carriers, excipients and adjuvants are added to the composition or formulation for a variety of purposes. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, PA.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 M to about 0.1 M and preferably 0.05 M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid pharmaceutical compositions can optionally have different types of coating. Coatings can be applied to the entire dosage form (e.g. a tablet) or a component of a dosage form (e.g., core, granules, beads, pellets, microparticles, etc). A coating can be used to improve patient compliance (e.g., taste-masking coating, flavor coating, coating to provide smooth surface for easy swallowing), to improve the stability of the compositions (e.g., protection from light, moisture, gas, acid protection, or to divide different layers or compartments to avoid a drug from interacting with different ingredients in a different layer/compartment), alter release profile of the drug (e.g., enteric coating, pH-dependent polymer coating, etc), or improve cosmetic considerations.

A coating can be a thin film-coating comprising one or more polymers or water soluble materials including but are not limited to, hypromellose, macrogol, hydroxyethylcellulose, sodium carboxymethylcellulose, polyethylene glycol, polyvinyl alcohol, and cellulose acetate phthalate. In addition, film coating can comprise one or more pharmaceutically acceptable excipients, including but not limited to titanium dioxide, ferric oxide, coloring agents, talc, or lecithin.

A coating that modifies release of the active ingredient can comprise a pH-dependent polymer (e.g., enteric polymer) or a pH-independent polymer. A release-modifying coating can comprise one or more polymers selected from methacrylic copolymers, aminoalkyl methacrylate copolymers, methacrylate copolymers, or ammonioalkyl methacrylate copolymers. A release-modifying coating can comprise one or more cationic polymer, anionic polymer, or neutral polymer.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared where the solid or amorphous components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, a pharmaceutical composition of the present disclosure is prepared for oral administration. In certain of such embodiments, a pharmaceutical composition is formulated by combining one or more agents and pharmaceutically acceptable carriers. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In certain embodiments, penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In other embodiments, the compounds of the present disclosure are administered by the intravenous route. In further embodiments, the parenteral administration may be provided in a bolus or by infusion.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more therapeutically active agents, or a pharmaceutically acceptable salt or solvate thereof are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances, the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In various aspects, the androgen receptor modulators in the pharmaceutical composition or combination as disclosed herein can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The compounds or pharmaceutical compositions of the present disclosure may be manufactured and/or administered in single or multiple unit dose forms.

Having now generally described the invention, the same will be more readily understood through reference to the following examples, which are provided by way of illustration and are not intended to be limiting of the present invention.

### EXAMPLES

The disclosure now being generally described, it will be more readily understood by reference to the following examples which are included merely for purposes of illustration of certain aspects and embodiments of the present invention, and are not intended to limit the invention.

### Example 1: Phase 1/2, Open-Label Study of the Combination of Enzalutamide and Compound A in Subjects with mCRPC

This portion of the study will primarily evaluate the safety and tolerability of the drug combination and establish the recommended doses for Compound A and enzalutamide when dosed in combination. In addition, blood sampling will be conducted for PK evaluation to assess the potential drug-drug interaction (DDI) between the two drugs.

In the Phase 1 part of the study each dose cohort will receive a 7-day run-in with single agent Compound A to establish steady-state concentrations of this drug at that dose level, followed by 28-day cycles of the combination Compound A with enzalutamide. Subjects may remain on study treatment as long as they are tolerating treatment without disease progression based on Response Evaluation Criteria in Solid Tumors (RECIST) v1.1 or based on Prostate Cancer Working Group 3 (PCWG3) criteria.

The Phase 1 part of the study each dose cohort will receive a 7-day run-in with single agent Compound A to establish steady-state concentrations of this drug at that dose level, followed by 28-day cycles of Compound A with a fixed dose of enzalutamide (120 mg/day). Compound A is evaluated at escalating doses along with the fixed dose of enzalutamide (120 mg/day); 1-4 dose levels of Compound A are as follows: 200 mg, 400 mg, 600 mg or 800 mg. The study allows 3 subjects to be enrolled in each cohort sequentially until a maximum tolerated dose or recommended dose is reached.

Compound A is administered once a day at 200 mg, 400 mg, 600 mg or 800 mg, i.e., 1-4 oral tablets comprising 200 mg of Compound A alone on days 1-7. Compound A is then administered once a day 200 mg, 400 mg, 600 mg or 800 mg, i.e., 1-4 oral tablets comprising 200 mg of Compound A in combination with a daily oral dosing of 120 mg of enzalutamide by an oral capsule in 28 day cycles. Enzalutamide will initially be administered as a once daily dose of three 40 mg capsules (120 mg/day), and the dose may be increased to four 40 mg capsules (160 mg/day).

The primary objectives of Phase 1 of this study are: To determine the safety and tolerability, maximum tolerated dose (MTD) and/or recommended Phase 2 combination dose (RP2CD), and DLTs of Compound A when administered in combination with a fixed dose of enzalutamide in subjects with mCRPC naïve to second generation anti-androgens, and to establish the recommended dose of enzalutamide when used in combination with Compound A in this subject population. This study is also to evaluate the PK of Compound A when dosed alone and in combination with enzalutamide in this subject population, and to evaluate the PK of enzalutamide when dosed in combination with Compound A in this subject population. Lastly, the objective of this study is to evaluate preliminary antitumor activity of Compound A in combination with enzalutamide in this subject population.

### Conclusion

After 7+ days and at least one cycle of 28 days, the study demonstrates that there is no DDI effect of Compound A on enzalutamide PK and the drug combination is both safe and tolerable. The study demonstrates that there are no serious adverse events (SAE) and specifically no TEAEs of Grade≥3 and no TESAEs of Grade≥3. Also noted is the antitumor activity in subjects with mCRPC when treated with all dose combinations.

### Example 2: Phase 2: Two Arm Randomized

Once the RP2CD for each drug has been established in Example 1, Phase 2 of the study will commence. Phase 2 is a two-arm, randomized (2:1), open-label study. Approximately 120 subjects with mCRPC will be randomized as follows: Group 1: Compound A at the daily dose of 200 mg, 400 mg, 600 mg or 800 mg per day in combination with enzalutamide at 120 mg/day or 160 mg/(n=80); Group 2: Enzalutamide single agent at 160 mg/day (n=40).

The planned dose of enzalutamide and Compound A for the combination arm will be those determined in the Phase 1 of this study based on safety and exposure data. Subjects may remain on study treatment so long as they are tolerating treatment without disease progression based on RECIST v1.1 and/or PCWG3 with up to at least up to 24 weeks of treatment. A 30-day End of Treatment visit will be conducted 30 days from the date of the last dose.

Subjects will be monitored for safety and clinical benefit. Additional PK data for both drugs will be collected. Evaluations of disease status will include computed tomography (CT)/magnetic resonance imaging (MRI) scans, bone scans, and measurements of prostate specific antigen (PSA) levels. During Phase 2 of the study, the SRB will meet to review all available data after 30, 60, and 90 subjects have completed 3 months of follow-up.

This study evaluates the antitumor activity of Compound A in combination with a fixed dose of enzalutamide compared with enzalutamide as a single agent in subjects with mCRPC naïve to second generation anti-androgens and to evaluate the safety of Compound A in combination with a fixed dose enzalutamide compared with enzalutamide as a single agent in this subject population. This study also evaluates the PK of Compound A when dosed in combination with a fixed dose enzalutamide in this subject population and the PK of enzalutamide when dosed in combination with Compound A in this subject population.

The conclusion of this study shows and establishes that at least 80% of subjects achieve a ≥50% reduction in PSA values from baseline at week 12. This study also establishes that at least 55% subjects achieve a ≥90% reduction in PSA values from baseline at week 24.

The study also demonstrates that there is no DDI effect of Compound A on enzalutamide and all drug combinations are both safe and tolerable. The study demonstrates that there are no serious adverse events (SAE) and specifically no TEAEs and TESAEs of Grade≥3, and antitumor activity in subjects with mCRPC when treated with all dose combinations.

### Example 3: Phase 1/2, Open-Label Study of the Combination of Enzalutamide and Compound A in Subjects with mCRPC—Phase 1

### Phase 1: Single Arm Dose Escalation (3+3 Dose Escalation Design)

The primary objectives of Phase 1 of this study are: To determine the safety and tolerability, maximum tolerated dose (MTD) and/or recommended Phase 2 combination dose (RP2CD), and dose-limiting toxicities (DLTs) of Compound A when administered in combination with a fixed dose of enzalutamide in subjects with mCRPC naïve to second generation anti-androgens, and to establish the RP2CD of enzalutamide when used in combination with Compound A in this subject population. This study is also to evaluate the PK of Compound A when dosed alone and in combination with enzalutamide in this subject population, and to evaluate the PK of enzalutamide when dosed in combination with Compound A in this subject population. Another objective of this study is to evaluate preliminary antitumor activity of Compound A in combination with enzalutamide in this subject population. This study aims to also measure changes in ctDNA levels in plasma and to characterize tumor aberration that are associated with response or resistance to Compound A and/or enzalutamide.

Phase 1 of the study is a single-arm dose escalation study of Compound A in combination with a fixed dose of enzalutamide (120 mg/day). This portion of the study primarily evaluates the safety and tolerability of the drug combination and establishes the RP2CD for Compound A and enzalutamide when dosed in combination. In addition, blood sampling will be conducted for PK evaluation to assess the potential drug-drug interaction (DDI) between the two drugs.

In the Phase 1 part of the study each dose cohort will receive a 7-day run-in with single agent Compound A to establish steady-state concentrations of this drug at that dose level, followed by 28-day cycles of the combination Compound A with enzalutamide. Subjects may remain on study treatment as long as they are tolerating treatment without disease progression based on Response Evaluation Criteria in Solid Tumors (RECIST) v1.1 or based on Prostate Cancer Working Group 3 (PCWG3) criteria. A 30-day End of Treatment Visit will be conducted 30 days from the date of the last dose.

Compound A is evaluated at escalating doses along with the fixed dose of enzalutamide (120 mg/day); 1-5 dose levels of Compound A are as follows: 200 mg, 400 mg, 600 mg, 800 mg, or 1000 mg. Additional Compound A dose levels, enzalutamide dose levels, and dose combinations can be added. The study allows at least 3 subjects to be enrolled in each cohort sequentially until a maximum tolerated dose or recommended phase 2 combination dose (RP2CD) is reached and additional about 12 subjects to be dosed at RP2CD.

Compound A is administered once a day (QD) or twice a day (BID) at 200 mg, 400 mg, 600 mg, 800 mg, or 1000 mg with or without food, i.e., 1-5 oral tablets per dose comprising 200 mg of Compound A alone on days 1-7 or 2-10 oral tablets per dose comprising 100 mg of Compound A alone on days 1-7, or a combination of 100 mg and 200 mg tablets. Compound A is then administered once a day or twice a day 200 mg, 400 mg, 600 mg, 800 mg or 1000 mg, i.e., 1-5 oral tablets per dose comprising 200 mg (or 2-10 tablets per dose comprising 100 mg or a combination with 200 mg tablets) of Compound A in combination with a daily oral dosing of 120 mg of enzalutamide by an oral capsule in 28 day cycles. Enzalutamide is initially administered as a once daily dose of three 40 mg capsules (120 mg/day), and the dose may be increased to four 40 mg capsules (160 mg/day).

Subjects may continue on treatment until objective or clinical disease progression develops, and/or occurrence of an unacceptable toxicity. Subjects enrolled in Phase 1 are allowed to escalate Compound A and/or enzalutamide sequentially to a higher dose cohort. Subjects may undergo more than 1 dose escalation of Compound A provided all escalation criteria have been met.

For Cohort 1, subjects (n=3) were orally administered 600 mg QD (once a day) of Compound A alone for 7 days then administered 600 mg QD Compound A in combination with 120 mg QD enzalutamide for at least one cycle (e.g., 28 days). Two subjects received the combination of Compound A and enzalutamide for at least 4 cycles (e.g., 16 weeks).

For Cohort 2, subjects (n=4) were orally administered 800 mg QD Compound A alone for 7 days then administered 800 mg QD Compound A in combination with 120 mg QD enzalutamide for at least one cycle.

For Cohort 3, subjects are orally administered 600 mg BID Compound A alone for 7 days then administer 600 mg BID Compound A in combination with 120 mg QD enzalutamide for at least one cycle.

MTD is defined according to the following standard criteria:

If none of the first 3 evaluable subjects in a dosing cohort experiences a protocol-defined DLT during the DLT assessment period, dose escalation will proceed with the next planned cohort.

If 1 of the first 3 evaluable subjects in a dosing cohort experiences a protocol-defined DLT during the DLT assessment period, an additional 3 subjects will be enrolled at the same dose level prior to escalating to the next dose.

If 1 of 6 subjects experiences a DLT during the DLT assessment period, the dose escalation will continue and 3 subjects will be enrolled in the next cohort.

If ≥2 of 6 subjects in a dosing cohort experience a DLT during the DLT assessment period, then the dose escalation will be stopped. This dose will then be considered an intolerable dose. The MTD will be determined as the dose of the next lower cohort. Additional cohorts of subjects at intermediate doses between the next lower dose cohort and the intolerable dose may be evaluated.

If ≥2 of 3 subjects in a dosing cohort experience a DLT during the DLT assessment period, dose escalation will be stopped. This dose will be considered an intolerable dose. An additional 3 subjects will then be added to the next lower cohort. If no additional DLTs are observed at that dose level then it will be determined to be the MTD. Additional cohorts of subjects at intermediate doses between the next lower dose cohort and the intolerable dose for either drug may be evaluated.

Once a RP2CD of Compound A in combination with a fixed dose of enzalutamide of 120 mg, an additional 3+3 cohort may also be included to explore a dose of 160 mg/day of enzalutamide in combination with a previously deemed safe dose of Compound A, depending on the results of safety and PK data regarding a potential DDI. The appropriate amount of follow up at the 120 mg/day dose that will be needed prior to potentially escalating to the 160 mg/day dose will be decided based on the totality of the PK and safety data available.

The DLT period for each subject in Phase 1 is through the end of Cycle 1 (defined from the start of the combination dosing up to Day 28 included). At least three consecutive subjects in a cohort must have received treatment through the DLT period without experiencing any DLTs before the opening of a new cohort at a higher dose level. Subjects who are not evaluable for safety throughout the DLT period for reasons other than Compound A-related toxicity, including due to disease progression, may be replaced in the same dose level cohort.

Based on safety data, including DLTs, and all available PK data, a new cohort can be added to the study at a higher dose level of either drug, expanding the current dose level, or dose de-escalating one or both drugs. While the primary basis for the dose level decisions will be the occurrence of DLTs, all available safety and PK data, including longer-term safety data from subjects treated in lower dose cohorts, will be considered. As DDIs are a possibility for this drug combination, doses may need to be adjusted for either drug based on PK results to achieve optimal plasma levels of both drugs.

The RP2CD for each drug is the MTD or a dose below the MTD which results in drug levels consistent with the anti-tumor activity in pre-clinical models and historical clinical data. Once the RP2CD for each drug is established, an additional 12 subjects may be added at the RP2CDs to further evaluate safety and PK.

DLTs are assessed for all subjects enrolled in Phase 1 starting the first day of combination dosing through the end of Cycle 1, Day 28 (the DLT period). Any adverse event (AE) occurring during the DLT period deemed at least possibly related to Compound A or the combination and meeting the criteria below will be designated a DLT.

### Non-Hematologic Toxicities

All non-hematologic Grade 4 AEs.

All non-hematologic Grade 3 AEs with the exception of the following:

Grade 3 events of nausea, vomiting, diarrhea, fatigue, or electrolyte abnormalities that resolve within 48 hours to baseline or ≤Grade 1 (resolution can be with or without medical management). An attempt must be made to manage these events before classifying them as a DLT.

Grade 3 amylase or lipase that is not associated with symptoms or clinical manifestations of pancreatitis.

Hy's Law, defined as aspartate aminotransferase (AST) or alanine aminotransferase (ALT)>3× the upper limit of normal (ULN) AND total bilirubin>2× ULN AND alkaline phosphatase<2× ULN AND no other reason for liver injury.

### Hematologic Toxicities

All Grade 4 hematologic toxicities.

Grade 3 neutropenic fever.

Grade 3 decrease of hemoglobin requiring blood transfusion.

Grade 3 or greater thrombocytopenia with significant bleeding.

Other events that qualify as a DLT

Any death that occurs during Cycle 1.

Treatment delay>15 days due to a related or possibly related AE with unresolved toxicity and failure to meet criteria for retreatment.

Any other clinically significant related AE if deemed to be a DLT by the Safety Review Board (SRB).

Inability to receive at least 75% of the planned dose due to an AE.

Any subject discontinuing the study in Phase 1 before the end of the first cycle (DLT assessment cycle) or who does not receive at least 75% of the planned dose for reasons other than drug-related safety may be replaced.

Inclusion Criteria—subjects are eligible for enrolling in the study if all of the following apply:

1. Institutional Review Board approved written informed consent and privacy language as per national regulations must be obtained for the subject or legally authorized representative prior to any study-related procedures being performed.

2. Males aged ≥18 years.

3. Histologically, pathologically, or cytologically confirmed prostate adenocarcinoma. In some instance, the inclusion is for prostate adenocarcinoma without small cell or neuroendocrine features (>10% small cell or neuroendocrine differentiation will be excluded).

4. Evidence of castration-resistant prostate cancer (CRPC) defined as PSA levels≥1 ng/mL while on androgen deprivation therapy (ADT) and documentation of 3 rising PSA levels taken at least 1 week apart during ADT (or surgical castration). This documentation can be from any time while the subject is on ADT.

5. Presence of metastatic disease at study entry documented by 1 or more bone lesions on bone scan or by soft tissue disease observed by CT/MRI.

6. Naïve to second generation anti-androgens. Previous exposure to docetaxel is allowed and will be used as a stratification factor.

7. Evidence of progressive disease defined as 1 or more Prostate Cancer Working Group 3 (PCWG3) criteria:
PSA≥1 ng/mL that has increased on at least 3 successive measurements taken at least 1 week apart;
Nodal or visceral progression as defined by RECIST 1.1 with the current PCWG3 recommendations;
Appearance of 2 or more new lesions in bone scan.

8. Eastern Cooperative Oncology Group (ECOG) performance status of 0 or 1.

9. Ongoing ADT with luteinizing hormone-releasing hormone (LHRH) agonist/antagonist therapy or history of bilateral orchiectomy, with castrate level testosterone.

10. Serum testosterone≤1.73 nmol/L (50 ng/dL).

11. Subjects receiving bisphosphonates or other approved bone-targeting therapy (e.g., denosumab) must be on a stable dose for at least 28 days prior to the start of study treatment.

12. Demonstrate adequate organ function as below:
a. Absolute neutrophil count>1500/μL, platelet count>100 000/μL; hemoglobin>5.6 mmol/L (9.0 g/dL) at screening. (Subjects must not have received any growth factors within 7 days or blood transfusions within 28 days prior to the hematologic laboratory values obtained at screening);
b. Total bilirubin<1.5×the upper limit of normal (ULN) at screening, except subjects with documented Gilbert's Syndrome who must have a total bilirubin<3 mg/dL;
c. Alanine aminotransferase and aspartate aminotransferase<2.5×ULN at screening;
d. Creatinine clearance≥45 mL/min and/or estimated glomerular filtration rate≥50;
e. Albumin>30 g/L (3.0 g/dL) at screening.

13. Subject of child-producing potential agree to use highly effective contraceptive methods and avoid sperm donation during the study treatment and for 3 months after the last dose of study treatment. A man is considered to be of child-producing potential unless he has had a bilateral vasectomy with documented aspermia or a bilateral orchiectomy.

14. All acute toxic effects of any prior treatment have resolved to NCI-CTCAE v5.0 Grade 1 or less.

15. Willing and able to comply with the protocol, including follow-up visits and examinations.

Exclusion Criteria—subjects are excluded from participation in the study if any of the following apply:

1. Biologic anti-cancer therapy (e.g., sipuleucel-T) within 28 days prior to the start of study treatment.

2. Use of hormonal agents with anti-tumor activity against prostate cancer including 5-alpha reductase inhibitors, androgens (e.g., testosterone), cytoproterone acetate, progestational agents, and estrogens/diethylstilbestrol within 28 days prior to the start of study treatment.

3. Use of herbal products or alternative therapies that may decrease PSA levels or that may have hormonal anti-prostate cancer activity (e.g., saw palmetto, PC-SPES, PC-HOPE, St. John's wort, selenium supplements, grape seed extract, etc.) within 28 days of study treatment initiation or plans to initiate treatment with these products/alternative therapies during the entire duration of the study.

4. Intervention with any chemotherapy, investigational agents, or other anti-cancer drugs within 28 days of the first dose of study treatment.

5. Use of radium-223 dichloride or other radioligand/radiopharmaceutical within 28 days prior to the start of study treatment.

6. Received limited-field palliative bone radiotherapy>5 fractions and/or any radiotherapy within 2 weeks prior to the start of study treatment (palliative radiation for bone pain is allowed).

7. Received a blood transfusion within 28 days of hematologic screening labs.

8. Known intra-cerebral disease or brain metastasis unless adequately treated and stable for the last 28 days before signing of informed consent.

9. Spinal cord compression as follows:
a. Any prior history of spinal cord compression related to prostate cancer (Treated spinal cord compression related to prostate cancer is allowed);
b. Any symptoms of neurologic compromise with radiographic evidence of potential spinal cord compression within 28 days prior to starting study treatment.

10. Diagnosis of another clinically significant malignancy within the previous 3 years other than curatively treated non-melanomatous skin cancer or superficial urothelial carcinoma and other in situ or non-invasive malignancies.

11. Gastrointestinal issues affecting absorption (e.g., gastrectomy).

12. Significant cardiovascular disease including any of the following:
a. Myocardial infarction within 6 months prior to signing informed consent;
b. Uncontrolled angina within 3 months prior to signing informed consent;
c. Congestive heart failure New York Heart Association (NYHA) class III or IV, or a history of congestive heart failure NYHA class III or IV unless a screening echocardiogram or multi-gated acquisition scan performed within 3 months prior to study entry results in a left ventricular ejection fraction that is <45%;
d. QT interval corrected by the Fridericia correction formula (QTcF)>480 msec at screening;
e. History of clinically significant ventricular arrhythmias (e.g., ventricular tachycardia, ventricular fibrillation, torsades de pointes);
f. History of Mobitz II second-degree or third-degree heart block;
g. Uncontrolled hypertension as indicated by a resting systolic blood pressure>160 mm Hg or diastolic blood pressure>90 mm Hg at screening.

13. Known history of seizure or conditions that may pre-dispose them to seizure, including brain injury with loss of consciousness, transient ischemic attack within the past 12 months, cerebral vascular accident, brain metastases, and brain arteriovenous malformation.

14. Concurrent disease or any clinically significant abnormality following the Investigator's review of the screening physical examination findings, 12-lead ECG results, and clinical laboratory tests, which in the judgment of the Investigator and/or sponsor would interfere with the subject's participation in this study or evaluation of study results.

15. Known or suspected hypersensitivity to any components of the formulation used for Compound A or enzalutamide.

16. Use of strong inhibitors of CYP2C8 (such as Gemfibrizol).

17. Use of strong inducers of CYP3A (such as Rifampin).

In some instances subjects are excluded if using compounds known to be strong inducers and strong inhibitors of CYP3A and CYP2C8 within 14 days of the first dose of study treatment.

In some instances subjects are excluded if using narrow therapeutic index sensitive CYP2C8 substrates (e.g., daprobustat, dasabuvir, repaglinide, paclitaxel), or sensitve substrates for CYP3A (alfentanil, avanafil, buspirone, conivaptan, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil) and CYP2B6 (bupropion) within 14 days prior to the first dose of study treatment.

In some instances subjects are excluded if using narrow therapeutic index drugs that are metabolized by CYP3A4 (e.g., alfentanil, cyclosporine, dihydroergotamine, ergotamine, fentanyl, pimozide, quinidine, sirolimus and tacrolimus), CYP2C9 (e.g., phenytoin, warfarin) and CYP2C19 (e.g., S-mephenytoin, clopidogrel) should be avoided, as enzalutamide may decrease their exposure. If co-administration with warfarin cannot be avoided, conduct additional international normalized ratio monitoring.

18. Use of granulocyte colony stimulating factor within 7 days prior to screening laboratories.

19. Ongoing participation in another therapeutic trial or use of another investigational agent within 28 days prior to the first dose of study treatment.

20. For Phase 2 subjects (see Example 2), prior participation in Phase 1.

21. Unwillingness or inability to comply with procedures required in this protocol.

22. Not a candidate for enzalutamide treatment, in the opinion of the Investigator.

In some instances, subject with rare hereditary problems of fructose intolerance will be excluded.

Phase 1 Discontinuation Criteria—the following events results in the removal of subjects from therapy:

1. Occurrence of a protocol-defined DLT.

2. Protocol-defined clinical or radiologic disease progression.

PSA progression is defined as a PSA level increase of ≥25% and >1 ng/mL above the nadir. A second confirmatory PSA measurement meeting the same criteria performed 3 or more weeks later is required. Subjects who only have PSA progression (without radiologic or clinical progression) and/or who are continuing to derive some clinical benefit per Investigator's assessment and have an acceptable safety profile may continue to receive treatment.

Clinical disease progression is defined as a skeletal-related event, or an event due to metastatic prostate cancer requiring intervention (e.g., a malignant pleural effusion). Skeletal-related events are defined as the following events due to bone metastatic prostate cancer:
Radiation therapy to bone metastases (external beam or injected radionuclides), with the exception of short course palliative radiation therapy due to pain and not disease progression,
Pathologic fracture, with the exception of fractures occurring as a result of increased physical activity associated with significant pain palliation,
Spinal cord compression,
Surgery to bone to treat or prevent complications of metastatic bone disease (e.g., stabilization of a pathologic fracture, decompression of a spinal cord compression).

Radiologic disease progression is defined as 2 new metastatic bone lesions on bone scan with confirmatory scan or soft tissue progression on CT/MRI per Response Evaluation Criteria in Solid Tumors (RECIST) v.1.1.

3. Any subject who does not restart treatment within 15 days of a dose interruption, with the exception of those who have a dose delay due to an elective procedure. If dose is delayed because of an elective procedure, no more than 28 days should elapse before the next dose.

4. Initiation of new anti-neoplastic therapy (excludes steroids or change in LHRH therapy).

5. Discontinuation of LHRH therapy (unless subject had a prior bilateral orchiectomy).

6. Gross study noncompliance by the subject as determined by the Investigator and/or Sponsor.

7. Determination by the Investigator in consultation with the Medical Monitor that continuing treatment is not in the best interest of the subject.

8. The Sponsor elects to end the study, or any portion thereof, for any reason.

### Allowed Therapies—Concomitant Medication

1. LHRH analogues are required if the subject has not had surgical castration. There are no restrictions on changing analogue or analogue dose during the study.

2. Short-course or taper (less than or equal to 2 weeks) of systemic steroids are permitted at the discretion of the Investigator to treat an AE.

3. Use of systemic corticosteroids to treat pain from metastatic bone disease or manage acute AEs is allowed during treatment.

4. Antiemetics and anti-diarrheal medications are allowed prophylactically according to standard clinical practice if clinically indicated.

5. Granulocyte colony stimulating factor is allowed if used per clinical guidelines (e.g., American Society of Clinical Oncology or European Society for Medical Oncology guidelines) but should not be used to meet eligibility required for hematologic laboratory parameters and cannot be used within 7 days of screening laboratories.

6. Drugs used to control bone loss (e.g., bisphosphonates and denosumab) are allowed if started at least 28 days prior to the start of study treatment and continued on a stable dose throughout the DLT period for Phase 1. For subjects in Phase 1, they may not be changed or initiated during the DLT period but may be stopped per standard of care. Following the DLT period in Phase 1, and for Phase 2 (see Example 2), these drugs may be added or changed.

7. Transfusions are allowed post-initiation of study treatment but not within 28 days prior to hematologic screening labs.

8. Opiate and non-opiate pain medication are allowed per standard of care.

Not-allowed therapies include concomitant medications that prolong the QT interval (QT) and/or the corrected QT interval (QTc). In addition, certain strong CYP2C8 inhibitors and certain strong CYP3A4 inhibitors or inducers, certain narrow therapeutic index sensitive CYP2C8, CYP3A4, CYP2C9, and CYP2C19 substrates, or certain sensitive substrates for CYP3A and CYP2B6, and certain medications associated with lowering the seizure threshold should not be used during the trial.

Study Endpoints—The primary variables for Phase 1 of the study include:

The incidence of protocol-defined DLTs during the DLT assessment period (first cycle). The DLTs will be characterized by type, frequency, severity (as graded by National Cancer Institute Common Terminology Criteria for AEs [NCI CTCAE version 5.0]), timing in relation to study treatment administration, seriousness, and relationship to study treatment.

Treatment-emergent adverse events (TEAEs; characterized by type, frequency, severity, timing, seriousness, and relationship to study treatment).

Abnormalities in clinical laboratory parameters, vital sign measurements, and electrocardiograms (ECGs) (characterized by type, frequency, severity, timing, seriousness, and relationship to study treatment).

Changes in ECOG performance status.

Secondary variables for Phase 1 include:

The plasma concentration data for Compound A, enzalutamide, N-desmethylated enzalutamide (M2; active metabolite) will be analyzed using non-compartmental methods to obtain estimates of standard PK parameters.

PK parameters of Compound A following multiple dosing including but not limited to plasma area under the concentration-time curve from time zero to 24 hours (AUC₀₋₂₄), maximum concentration (Cₘₐₓ), observed pre-dose plasma concentration during multiple dosing (C_{trough}), time to reach Cₘₐₓ (Tₘₐₓ), apparent terminal elimination half-life (t½; whenever feasible to calculate), apparent volume of distribution at steady state after extravascular administration (Vₛₛ/F), apparent clearance after extravascular administration (CL/F).

PK parameters of enzalutamide and its primary active metabolite following multiple dosing including but not limited to AUC₀₋₂₄, Cₘₐₓ, C_{trough}, Tₘₐₓ, t½ (whenever feasible to calculate), Vₛₛ/F, CL/F.

Exploratory variables in Phase 1 include:

Changes from baseline in PSA blood concentrations at the end of each cycle.

Objective response as measured by RECIST v.1.1 (in subjects with measurable disease) according to PCWG3 guidelines.

Duration of response (DOR).

To measure changes in ctDNA levels in plasma and to characterize tumor aberration.

### Safety Analysis: Conclusion of Cohort 1

Cohort 1 subjects cleared the DLT period with no grade 3 treatment-related adverse events. The safety profile for the combination was consistent with second generation anti-androgens, such as grade 1 or 2 adverse events of fatigue and hot-flashes.

PK analysis of Cohort 1 showed that enzalutamide exposure was only minimally impacted by Compound A administration while, Compound A exposure was impacted by enzalutamide with higher metabolism of Compound A observed.

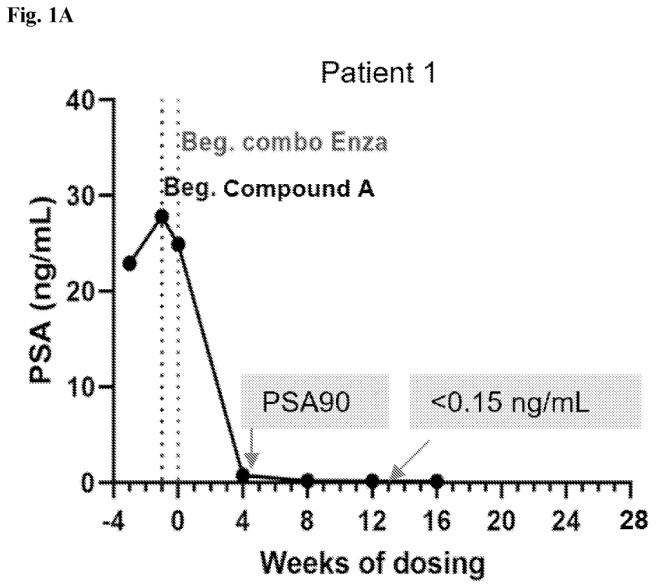

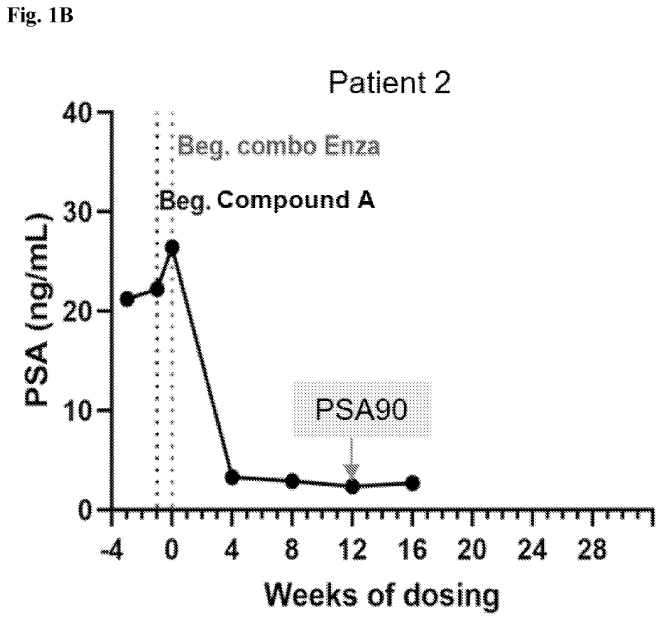

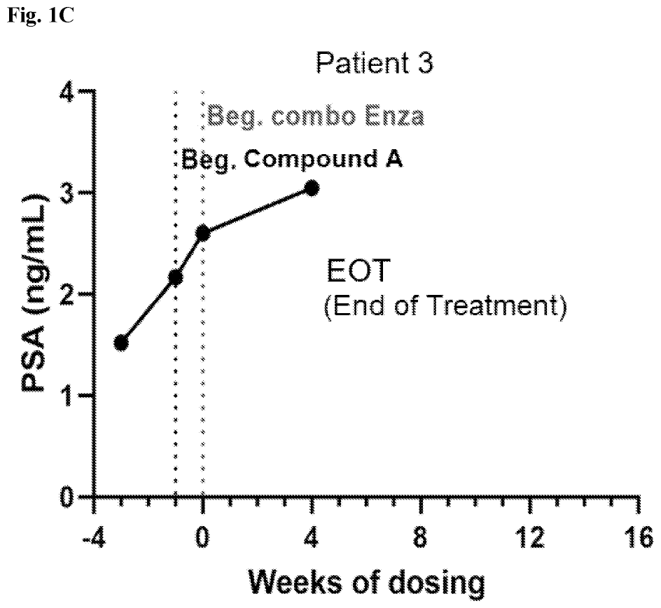

Changes in PSA levels in the three subjects from Cohort 1 is shown in FIGS. 1A-1C. Two of the three subjects achieved a rapid decline in the prostate-specific antigen (PSA) level by week 4 and a PSA90 (PSA decline of >90%) within three months of starting combination treatment (FIGS. 1A-1B). Thus, PSA90 response rate for Cohort 1 was about 67%. One subject with no prior chemotherapy achieved PSA<0.2 ng/mL within three months (FIG. 1A)—<0.2 ng/mL rate of 33%. In enzalutamide monotherapy trials, 90 day confirmed PSA<0.2 ng/mL rates were 5% and 1% for pre-chemotherapy population and post-chemotherapy population, respectively.

The third subject was discontinued from the study at the end of cycle 2 due to drug-drug interaction between a concomitant medication (strong CYP3A inducer) with Compound A and enzalutamide, resulting in a significant decrease in exposure of both Compound A and enzalutamide (FIG. 1C).

For the first subject who had no prior chemotherapy (FIG. 1A), AUCss for Compound A was approximately 235,000 ng·hr/mL and AUCss for enzalutamide and its active metabolite N-desmethyl enzalutamide (M2 metabolite) was approximately 540,000 ng·hr/mL. The second subject, who received prior chemotherapy (FIG. 1B), demonstrated AUCss for Compound A of approximately 62,000 ng·hr/mL and AUCss for enzalutamide+M2 metabolite of approximately 430,000 ng·hr/mL. The third subject, who also received prior chemotherapy (FIG. 1C), demonstrated AUCss for Compound A of approximately 33,000 ng·hr/mL and AUCss for enzalutamide+M2 metabolite of approximately 390,000 ng·hr/mL.

Radiographic assessment in first and second subjects (FIGS. 1A-1B) at 8 weeks and 16 weeks shoed stable disease (bone only).

### Safety Analysis: Conclusion of Cohort 2

Three of the four Cohort 2 subjects cleared the DLT period with no grade 3 treatment-related adverse events. The fourth Cohort 2 subject has not completed the DLT period yet. Thus far, the safety profile for the combination was consistent with second generation anti-androgens, such as grade 1 or 2 adverse events of fatigue and hot-flashes.

### Example 4: Phase 1/2, Open-Label Study of the Combination of Enzalutamide and Compound A in Subjects with mCRPC—Phase 2

### Phase 2: Two Arm Randomized

Once the RP2CD for each drug has been established in Example 3, Phase 2 of the study will commence. Phase 2 is a two-arm, randomized (2:1), open-label study. Approximately 120 subjects with mCRPC will be randomized as follows: Group 1: Compound A at the daily dose of 200 mg, 400 mg, 600 mg, 800 mg, or 1000 mg per day or the RP2CD as determined in Example 3 in combination with enzalutamide at 120 mg/day or 160 mg/day (n=80); Group 2: Enzalutamide single agent at 160 mg/day (n=40). Subjects will orally take Compound A and enzalutamide at approximately the same time each day with or without food. Subjects will receive study treatment(s) in 28-day cycles. Subjects may continue on treatment until objective or clinical disease progression develops, occurrence of an unacceptable toxicity, or if any discontinuation criteria are met.

The planned dose of enzalutamide and Compound A for the combination arm will be those determined in the Phase 1 of this study based on safety and exposure data. Subjects may remain on study treatment so long as they are tolerating treatment without disease progression based on RECIST v1.1 and/or PCWG3. A 30-day End of Treatment visit will be conducted 30 days from the date of the last dose.

Subjects will be monitored for safety and clinical benefit. Additional PK data for both drugs will be collected. Evaluations of disease status will include computed tomography (CT)/magnetic resonance imaging (MRI) scans, bone scans, and measurements of prostate specific antigen (PSA) levels. During Phase 2 of the study, the SRB will meet to review all available data after 30, 60, and 90 subjects have completed 3 months of follow-up.

This study evaluates the antitumor activity of Compound A in combination with a fixed dose of enzalutamide compared with enzalutamide as a single agent in subjects with mCRPC naïve to second generation anti-androgens and to evaluate the safety of Compound A in combination with a fixed dose enzalutamide compared with enzalutamide as a single agent in this subject population. This study also evaluates the PK of Compound A when dosed in combination with a fixed dose enzalutamide in this subject population and the PK of enzalutamide when dosed in combination with Compound A in this subject population.

Inclusion and Exclusion Criteria: Allowed and Non-Allowed Therapies (concomitant medication)—see Example 3.

Phase 2 Discontinuation Criteria—The discontinuation criteria for subjects in Phase 2 are the same as Phase 1 (see Example 3) with the following clarification for discontinuation due to AEs:

Occurrence of a toxicity which fits the definition of a protocol-defined DLT occurring anytime during drug treatment (i.e., not limited to the first cycle) requires discontinuation with the following exception:
Grade 3 related or possibly related AEs do not require discontinuation if the AE(s) resolve to baseline or Grade 1 within 7 days with adequate medical management, dose interruption and/or dose reductions, and restart of study treatment.

Subjects who discontinue prior to receiving any dose of Compound A or enzalutamide in Phase 2 may be replaced.

Study Endpoints—The primary variables for Phase 2 of the study is the proportion of subjects with a prostate-specific antigen decline of >50% (PSA50) at Week 12.

Additional efficacy variables for Phase 2 include:

Proportion of subjects with PSA90 at Week 12 and Week 24.

Proportion of subjects with PSA50 at Week 24.

Time to PSA progression, as described in PCWG3.

Time to radiographic progression using the RECIST v 1.1 definition.

Objective response rate as measured by RECIST v.1.1 (in subjects with measurable disease) according to PCWG3 guidelines.

DOR.

Progression-free survival (PFS).

The safety variables in Phase 2 include:

TEAEs

Abnormalities in clinical laboratory parameters, vital sign measurements, and ECGs

Changes in ECOG performance status

The PK endpoints in Phase 2 include:

PK parameters of Compound A following multiple dosing, including but not limited to plasma AUC0-24, Cmax, Ctrough, Tmax, t½ (whenever feasible to calculate), Vss/F, and CL/F.

PK parameters of enzalutamide and its primary active metabolite following multiple dosing, including but not limited to plasma AUC₀₋₂₄, Cₘₐₓ, C_{trough}, Tₘₐₓ, t½ (whenever feasible to calculate), Vₛₛ/F, and CL/F.

### Safety Analysis—see Example 3.

Efficacy Analysis: Efficacy analysis will be performed on all subjects in the efficacy set, unless specified otherwise. All efficacy analyses are conducted in Phase 2 of the study. Binary endpoints will be displayed with their exact Clopper-Pearson binomial 90% confidence intervals. Time-to-event endpoints will be estimated using Kaplan-Meier techniques. The primary efficacy endpoint occurs in Phase 2 of the study and is the proportion of subjects with a prostate-specific antigen decline of >50% (PSA50) at Week 12. Unless otherwise specified, subjects with missing or no post-baseline PSA assessments will be considered non-responders. A one-sided Cochran-Mantel-Haenszel (CMH) test will be used to analyze the PSA response between treatment arms accounting for the stratification of prior docetaxel exposure. An odds ratio of PSA response in the Compound A+enzalutamide group to the PSA response in the enzalutamide single agent group will be presented with 90% confidence intervals. The Chi-square statistic and associated p-value for the CMH test will also be presented to determine if there is any significant association between PSA response and treatment arm while controlling for previous docetaxel exposure. Secondary endpoints including time to radiographic progression and objective response will be assessed according to RECIST v1.1. Time to PSA progression will be assessed as described in PCWG3. Additional responder analyses will be defined as above using a post-baseline PSA decline>90% as the responder cutoff. Objective response rate (ORR) will be calculated for subjects with measurable disease at baseline for each treatment arm along with exact Clopper-Pearson 90% confidence intervals (Cis). The difference in ORR between treatment arms will also be calculated and presented alongside exact 90% CIs. Progression-free survival (PFS) and 95% CIs will be presented.

Overall response rates will be summarized using frequencies and percentages of subjects with a best overall response of complete response (CR), partial response (PR), stable disease (SD), progressive disease (PD) or not evaluable (NE) based on RECIST v1.1 criteria. DoR is defined as the time from first documentation of response (CR, PR, or PSA reduction by ≥50%) until first documentation of confirmed progressive disease.

### Pharmacokinetic Analyses—see Example 3.

The conclusion of this study shows and establishes that at least 80% of subjects achieve a ≥50% reduction in PSA values (PSA50 response) from baseline at week 12. This study also establishes that at least 55% subjects achieve a ≥90% reduction in PSA values from baseline at week 24.

The study also demonstrates that there is no DDI effect of Compound A on enzalutamide and all drug combinations are both safe and tolerable. The study demonstrates that there are no serious adverse events (SAE) and specifically no TEAEs and TESAEs of Grade≥3, and antitumor activity in subjects with mCRPC when treated with all dose combinations.

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A pharmaceutical combination comprising a therapeutically effective amount of a first therapeutically active agent Compound A having the structure: 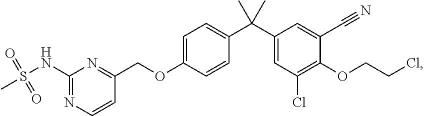 [SMILES: CC(C)(c1ccc(OCc2ccnc(NS(C)(=O)=O)n2)cc1)c1cc(Cl)c(OCCCl)c(C#N)c1]
or a pharmaceutically acceptable salt or solvate thereof, and enzalutamide in at least one pharmaceutical composition.

2. The pharmaceutical combination of claim 1, wherein the combination of Compound A and enzalutamide are in at least two pharmaceutical compositions.

3. The pharmaceutical combination of claim 2, wherein the at least two pharmaceutical compositions are at least two different dosage forms.

4. The pharmaceutical combination of claim 2 or claim 3, wherein the at least two pharmaceutical compositions or dosage forms are co-packaged together into a single kit.

5. The pharmaceutical combination of any one of claims 1-4, wherein a daily dosage amount of Compound A is between about 50 mg and about 1500 mg, or between about 100 mg and about 1000 mg, or between about 200 mg and about 800 mg, or between about 300 mg and about 600 mg.

6. The pharmaceutical combination of any one of claims 1-5, wherein Compound A is included in at least one tablet and the amount of Compound A per tablet is between about 5 mg and about 1000 mg, or between about 10 mg and about 500 mg, or between about 20 mg and about 400 mg, or between about 75 mg and about 300 mg, or between about 150 mg and about 250 mg.

7. The pharmaceutical combination of any one of claims 1-6, wherein Compound A is included in at least one tablet and amount of Compound A per tablet is about 200 mg.

8. The pharmaceutical combination of any one of claim 1-7, wherein a daily dosage amount of enzalutamide is about 20 mg to about 500 mg, or about 40 mg to about 250 mg, or about 75 mg to about 300 mg, or about 100 mg to about 200 mg, or about 110 mg to about 170 mg.

9. The pharmaceutical combination of any one of claim 1-8, wherein a daily dosage amount of enzalutamide is about 120 mg or about 160 mg.

10. The pharmaceutical combination of any one of claims 1-9, wherein the amount of enzalutamide per a dosage is about 20 mg to about 500 mg, or about 40 mg to about 250 mg, or about 75 mg to about 300 mg, or about 100 mg to about 200 mg, or about 110 mg to about 170 mg.

11. The pharmaceutical combination of claim 10, wherein the amount of enzalutamide per dosage is about 120 mg or about 160 mg.

12. The pharmaceutical combination of claim 11, wherein the about 120 mg or about 160 mg dosage of enzalutamide is provided in at least three pharmaceutical compositions.

13. The pharmaceutical combination of any one of claims 1-11, wherein a pharmaceutical composition of enzalutamide comprises about 5 mg to about 200 mg enzalutamide, or about 20 mg to about 100 mg enzalutamide, or about 30 mg to about 80 mg enzalutamide, or about 40 mg to about 60 mg enzalutamide.

14. The pharmaceutical combination of claim 13, wherein the pharmaceutical composition comprises about 40 mg of enzalutamide.

15. The pharmaceutical combination of claim 14, wherein the composition comprising enzalutamide is an oral capsule.

16. The pharmaceutical combination of any one of claims 1-15, wherein Compound A and enzalutamide and are in the same composition.

17. The pharmaceutical combination of any one of claims 1-16, wherein Compound A and enzalutamide are in different pharmaceutical compositions as a kit.

18. The pharmaceutical combination of claim 17, wherein the kit comprises 1 to 6 compositions for each therapeutically active agent to be administered per day.

19. The pharmaceutical combination of any one of claims 1-18, wherein at least one composition is a tablet or a capsule.

20. The pharmaceutical combination of any one of claims 1-19, when administered to a subject, do not produce any serious adverse event to the subject.

21. The pharmaceutical combination of any one of claims 1-19, when administered to a subject, does not produce a treatment-emergent adverse event (TEAE) or a treatment-emergent serious adverse event (TESAE).

22. A method for treating cancer, comprising administering the pharmaceutical combination of any one of claims 1-21 to a subject in need thereof.

23. The method of claim 22, wherein the cancer is selected from prostate cancer, breast cancer, ovarian cancer, bladder cancer, pancreatic cancer, hepatocellular cancer, endometrial cancer, or salivary gland carcinoma.

24. The method of claim 23, wherein the cancer is prostate cancer.

25. The method of claim 24, wherein the prostate cancer is primary or localized prostate cancer, locally advanced prostate cancer, recurrent prostate cancer, advanced prostate cancer, metastatic prostate cancer, metastatic castration-resistant prostate cancer, non-metastatic castration-resistant prostate cancer, and hormone-sensitive prostate cancer.

26. The method of claim 25, wherein the prostate cancer is metastatic castration-resistant prostate cancer.

27. The method of claim 26, wherein the prostate cancer expresses full-length androgen receptor or truncated androgen receptor splice variant.

28. The method of any one of claims 22-27, wherein the administration of the pharmaceutical combination does not produce any serious adverse event to the subject.

29. The method of any one of claims 22-27, wherein the administration of the pharmaceutical combination does not produce a treatment-emergent adverse event (TEAE) or a treatment-emergent serious adverse event (TESAE) to the subject.
